(19) United States
(12) Patent Application Publication
(10) Pub. No.: US 2024/0091240 A1
(43) Pub. Date: Mar. 21, 2024

(54) TREATMENTS FOR BLOOD-BRAIN BARRIER DYSFUNCTION AND RECURRENT SEIZURES USING NOX/LOX/COX INHIBITORS

(71) Applicant: University of Kentucky Research Foundation, Lexington, KY (US)

(72) Inventors: Bjoern Bauer, Lexington, KY (US); Anika Maria Sophie Hartz, Lexington, KY (US)

(21) Appl. No.: 18/370,558

(22) Filed: Sep. 20, 2023

Related U.S. Application Data
(60) Provisional application No. 63/408,739, filed on Sep. 21, 2022.

(51) Int. Cl. A61K 31/635; A61K 31/198; A61K 31/381; A61K 31/407; A61P 25/08

(52) U.S. Cl. CPC A61K 31/635; A61K 31/198; A61K 31/381; A61K 31/407; A61P 25/08

(57) ABSTRACT

Methods for treating blood-brain barrier dysfunction and methods for treating recurrent seizures in a subject involve inhibiting nicotinamide adenine dinucleotide phosphate (NADPH) oxidase (NOX), 5-lipoxygenase (5-LOX), and cyclooxygenase-2 (COX-2).

### CROSS-REFERENCE TO RELATED APPLICATION

The present application claims priority to U.S. Patent Application Ser. No. 63/408,739, filed on Sep. 21, 2022, the entire disclosure of which is incorporated herein by reference.

### GOVERNMENT INTEREST

This invention was made with government support under grant number R01AG075583 awarded by the National Institute of Health. The government has certain rights in the invention.

### TECHNICAL FIELD

The presently disclosed subject matter generally relates to treatments for blood-brain barrier (BBB) dysfunction and treatments for recurrent seizures in subjects. In particular, certain embodiments of the presently disclosed subject matter relate to methods of treatment that involve inhibiting nicotinamide adenine dinucleotide phosphate (NADPH) oxidase (NOX), 5-lipoxygenase (5-LOX), and cyclooxygenase-2 (COX-2) in a subject.

### BACKGROUND

Epilepsy has long been recognized as a co-morbidity in Alzheimer's disease (AD). New evidence shows that early-stage AD patients experience seizures, and data from the most recent clinical studies demonstrate that as many as 65% of AD patients have seizures/epilepsy. These data indicate that epilepsy incidence in AD has long been underestimated and is much greater than originally reported. Despite the clinical relevance, researchers have mostly ignored epilepsy in AD, leaving clinicians with limited therapeutic options.

As a result, treatment of AD patients with epilepsy remains a challenge. AD and epilepsy share common pathologic hallmarks that contribute to disease progression. One such hallmark is blood-brain barrier (BBB) dysfunction.

BBB dysfunction can result in connection with vessel deformation, vascular leakage, vascular inflammation, altered clearance, changes to tight junction proteins, changes to metabolic enzymes, changes to signaling molecules, changes in leukocyte recruitment, and changes in transporter expression.

BBB dysfunction is associated with various conditions impacting the brain, such as epilepsy, seizure, Alzheimer's disease and/or dementia, Parkinson's disease, brain cancer, multiple sclerosis, stroke, brain trauma, infectious diseases of the brain, and peripheral inflammation or inflammation of the central nervous system (CNS).

BBB dysfunction is recognized as both a cause and a consequence of seizures in epilepsy, yet therapeutic options for restoring BBB function are limited. Published findings show that a dysfunctional BBB contributes to seizure genesis and promotes epilepsy progression. Traditionally, the standard of care for treatment of epilepsy has involved the use of pharmacotherapy with anti-seizure drugs (ASDs), which act inside the brain on neurons. However, about 30%-40% of patients do not respond to ASDs and are drug-resistant (refractory epilepsy). In this regard, BBB leakage is thought to affect ASD resistance due to the influx of serum proteins that can bind to ASDs and reduce their effectiveness in the brain. Even in patients who respond to ASDs, there are often adverse effects.

### SUMMARY

The presently disclosed subject matter meets some or all of the above-identified needs, as will become evident to those of ordinary skill in the art after a study of information in this document.

This Summary describes several embodiments of the presently disclosed subject matter and, in many cases, lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features or all embodiments disclosed herein.

The presently disclosed subject matter includes methods for treating blood-brain barrier (BBB) dysfunction in a subject, which involve inhibiting nicotinamide adenine dinucleotide phosphate (NADPH) oxidase (NOX), 5-lipoxygenase (5-LOX), and cyclooxygenase-2 (COX-2) in the subject. Inhibition of NOX, 5-LOX, and COX-2 is achieved by administering a combination of a NOX inhibitor and one or more inhibitors for inhibiting 5-LOX and COX-2 to the subject.

In some embodiments, the combination is administered in an effective amount to reduce BBB leakage in the subject. In some embodiments a reduction in BBB leakage is characterized by a reduction in brain capillary leakage in the subject. In some embodiments, the one or more inhibitors for inhibiting 5-LOX and COX-2 of the combination include a 5-LOX inhibitor and a COX-2 inhibitor. In some embodiments, the one or more inhibitors for inhibiting 5-LOX and COX-2 of the combination include a dual 5-LOX/COX-2 inhibitor. In some embodiments, the methods for treating BBB dysfunction further involve identifying the subject as having seizures, epilepsy, Alzheimer's disease and/or dementia, Parkinson's disease, brain cancer, multiple sclerosis, stroke, brain trauma, an infectious disease of the brain, and/or peripheral inflammation or inflammation of the central nervous system (CNS).

The presently disclosed subject matter also includes methods for treating recurrent seizures, which involve inhibiting NOX, 5-LOX, and COX-2 to a subject in need thereof. Inhibition of NOX, 5-LOX, and COX-2 is achieved by administering a combination of a NOX inhibitor and one or more inhibitors for inhibiting 5-LOX and COX-2 to the subject.

In some embodiments, the methods for treating recurrent seizures further involve identifying a subject as having Alzheimer's disease with epilepsy. In some embodiments, the one or more inhibitors for inhibiting 5-LOX and COX-2 of the combination includes a 5-LOX inhibitor and a COX-2 inhibitor. In some embodiments, the one or more inhibitors for inhibiting 5-LOX and COX-2 of the combination includes a dual 5-LOX/COX-2 inhibitor. In some embodiments, the methods for treating recurrent seizures further involve administering an anti-seizure drug to the subject.

### BRIEF DESCRIPTION OF THE DRAWINGS

The presently disclosed subject matter will be better understood, and features, aspects, and advantages other than those set forth above will become apparent when consideration is given to the following detailed description thereof. Such detailed description makes reference to the following drawings of which:

FIG. 1A includes a graph showing the results of a Texas Red (TR) leakage assay showing seizure-mediated capillary leakage in 5×FAD mice (male, 4 months) (5×FAD-Epi) administered a combination of NOX, 5-LOX, and COX-2 inhibitors (100/5/10 mg/kg NAC/ZLT/CEL) every 12 hours for 2 days as compared to wild-type (WT), seizure-free 5×FAD mice, and untreated 5×FAD-Epi mice receiving vehicle (n=8 mice/group). Seizure induction with kainic acid. Data mean±SEM; Statistics: *** p<0.001, * p<0.05 (ANOVA). In this assay, increased capillary leakage correlates with reduced luminal TR fluorescence; mannitol is a positive control for leakage.

FIG. 1B includes a bar graph showing S1000 ELISA (normalized) levels in serum from 5×FAD-Epi mice administered a combination of NOX, 5-LOX, and COX-2 inhibitors (100/5/10 mg/kg NAC/ZLT/CEL) every 12 hours for 2 days as compared to WT, seizure-free 5×FAD mice, and untreated 5×FAD-Epi mice receiving vehicle (n=8 mice/group). Seizure induction with kainic acid. Data mean±SEM; Statistics: *** p<0.001, * p<0.05 (ANOVA).

FIG. 2A includes a graph showing the results of a TR leakage assay showing seizure-mediated capillary leakage in TgF344-AD rats (female, 6 months) (TgF344-Epi) administered a combination of NOX, 5-LOX, and COX-2 inhibitors (100/5/10 mg/kg NAC/ZLT/CEL) every 12 hours for 2 days as compared to wild-type (WT), seizure-free TgF344 rats, and untreated TgF344-Epi rats receiving vehicle (n=6-8 rats/group). Seizure induction with pilocarpine. Data mean±SEM; Statistics: *** p<0.001, * p<0.05 (ANOVA). In this assay, increased capillary leakage correlates with reduced luminal TR fluorescence; mannitol is a positive control for leakage.

FIG. 2B includes a bar graph showing S1000 ELISA (normalized) levels in serum from TgF344-Epi rats administered a combination of NOX, 5-LOX, and COX-2 inhibitors (100/5/10 mg/kg NAC/ZLT/CEL) every 12 hours for 2 days as compared to WT, seizure-free TgF344 rats, and untreated TgF344-Epi rats receiving vehicle (n=6-8 rats/group). Seizure induction with pilocarpine. Data mean±SEM; Statistics: *** p<0.001, * p<0.05 (ANOVA).

### DESCRIPTION OF EXEMPLARY EMBODIMENTS

The details of one or more embodiments of the presently disclosed subject matter are set forth in this document. Modifications to embodiments described in this document and other embodiments will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently disclosed subject matter includes methods for treating blood-brain barrier (BBB) dysfunction in a subject in which a combination of a nicotinamide adenine dinucleotide phosphate (NADPH) oxidase (NOX) inhibitor and one or more inhibitors for inhibiting 5-lipoxygenase (5-LOX) and cyclooxygenase-2 (COX-2) is administered to the subject to reduce BBB leakage.

As will be recognized by those skilled in the art, BBB dysfunction is associated with one or more of vessel deformation, vascular leakage, vascular inflammation, altered clearance, changes to tight junction proteins, changes to metabolic enzymes, changes to signaling molecules, changes in leukocyte recruitment, and transporter overexpression. Examples of implicated transporters include, but are not necessarily limited to, P-glycoprotein (Pgp), Breast Cancer Resistance Protein (BCRP), Multidrug resistance-associated protein (MRP), Monocarboxylate transporter 1 (MCT1), L-Type Amino Acid Transporter 1 (LAT1), Organic anion-transporting polypeptide 2 (OATP2).

BBB dysfunction within a subject may be identified by an increase in BBB leakage in the subject as compared to another subject without BBB dysfunction or as compared to the subject prior to the onset of a condition contributing to an increase in BBB leakage. A reduction in BBB dysfunction within the subject may, in turn, be characterized by a reduction in BBB leakage within the subject. In some embodiments, a reduction in BBB leakage may be characterized by a reduction in brain capillary leakage. Of course, BBB dysfunction within a subject may be identified by any suitable means that would be known to those of skill in the art without departing from the spirit and scope of the present disclosure. For example, in some embodiments, BBB dysfunction within a subject may be identified by one or more of the other conditions identified above and commonly associated with BBB dysfunction besides BBB leakage, such as an increase in neurovascular inflammation as compared to a subject without BBB dysfunction. A reduction in BBB dysfunction can also be determined based on an alteration (e.g., a decrease or an increase) in one of the other conditions identified above and commonly associated with BBB dysfunction besides BBB leakage. Brain capillary leakage and reductions thereof can be determined utilizing techniques and analytical procedures that are well-known in the art.

In an exemplary method for treating BBB dysfunction in a subject, the subject is administered an effective amount of a combination comprising a NOX inhibitor, a 5-LOX inhibitor, and a COX-2 inhibitor (or NOX/5-LOX/COX-2 inhibitor combination) to reduce BBB leakage. Accordingly, in some embodiments, the one or more inhibitors for inhibiting 5-LOX and COX-2 of the combination administered to the subject comprises two separate inhibitors: a 5-LOX inhibitor and a COX-2 inhibitor. In some embodiments, the NOX/5-LOX/COX-2 inhibitor combination is administered to the subject in a single dosage form, i.e., the NOX inhibitor, the 5-LOX inhibitor, and the COX-2 inhibitor of the NOX/5-LOX/COX-2 inhibitor combination are administered as a combination drug. In other embodiments, the NOX/5-LOX/COX-2 inhibitor combination may be administered to the subject in a multi-unit dosage form in which the subject is administered multiple, separate compositions, with each composition including one or more of the NOX inhibitor, the 5-LOX inhibitor, and the COX-2 inhibitor of the NOX/5-LOX/COX-2 inhibitor combination. For instance, in some embodiments, the subject may be administered two separate compositions: a first composition comprising the 5-LOX inhibitor and the COX-2 inhibitor; and a second composition comprising the NOX inhibitor. In some embodiments, the subject may be administered three separate compositions: a first composition comprising the 5-LOX inhibitor; a second composition comprising the COX-2 inhibitor; and a third composition comprising the NOX inhibitor. In some embodiments, the NOX/5-LOX/COX-2 inhibitor combination is administered to the subject twice daily. In some embodiments, the NOX/5-LOX/COX-2 inhibitor combination is administered to the subject for at least two days

As disclosed in commonly assigned U.S. Pat. No. 11,433,052, which is incorporated herein in its entirety by reference, various 5-LOX inhibitors are known to those skilled in the art. Known 5-LOX inhibitors include, but are not necessarily limited to, zileuton (ZLT), meclofenamate sodium, baicalein, caffeic acid, curcumin, hyperforin, and acetyl-keto-beta-boswellic acid (AKBA). Various embodiments of the NOX/5-LOX/COX-2 inhibitor combination are contemplated herein, and each respective 5-LOX inhibitor identified above may be utilized in a different embodiment of the NOX/5-LOX/COX-2 inhibitor combination. Furthermore, embodiments of the NOX/5-LOX/COX-2 inhibitor combination utilized in the treatment methods described herein can include one or more 5-LOX inhibitors.

Various COX-2 inhibitors are also known to those skilled in the art and include, but are not necessarily limited to, celecoxib (CEL), valdecoxib, and rofecoxib. Various embodiments of the NOX/5-LOX/COX-2 inhibitor combination are contemplated herein, and each respective COX-2 inhibitor identified above may be utilized in a different embodiment of the NOX/5-LOX/COX-2 inhibitor combination. Furthermore, embodiments of NOX/5-LOX/COX-2 inhibitor combination utilized in the methods described herein can include one or more COX-2 inhibitors.

Various NOX inhibitors are also known to those skilled in the art and include, but are not necessarily limited to, diphenyleneiodonium, apocynin, honokiol, plumbagin, AEBSF, phenylarsine oxide, gilotoxin, ebselen, VAS2870, HMGCoA, and N-acetylcysteine (NAC). Various embodiments of the NOX/5-LOX/COX-2 inhibitor combination are contemplated herein, and each respective NOX inhibitor identified above may be utilized in a different embodiment of the NOX/5-LOX/COX-2 inhibitor combination. Furthermore, embodiments of the drug combinations utilized in the methods described herein can include one or more NOX inhibitors.

In some embodiments, the NOX inhibitor of the NOX/5-LOX/COX-2 inhibitor combination is N-acetylcysteine. In some embodiments, the 5-LOX inhibitor of the NOX/5-LOX/COX-2 inhibitor combination is zileuton. In some embodiments, the COX-2 inhibitor of the NOX/5-LOX/COX-2 inhibitor combination is celecoxib. In some embodiments, the NOX/5-LOX/COX-2 inhibitor combination comprises acetylcysteine, zileuton, and celecoxib. In some embodiments, administering an effective amount of the NOX/5-LOX/COX-2 inhibitor combination comprises administering N-acetylcysteine in a dosage of about 100 mg/kg, administering zileuton in a dosage of about 5 mg/kg, and administering celecoxib in a dosage of about 10 mg/kg.

In some embodiments, administration of the NOX/5-LOX/COX-2 inhibitor combination is effective in reducing brain capillary leakage in the subject.

In another exemplary method for treating BBB dysfunction in a subject, the subject is administered an effective amount of a combination comprising a NOX inhibitor and a dual 5-LOX/COX-2 inhibitor. Accordingly, in some embodiments, the one or more inhibitors for inhibiting 5-LOX and COX-2 of the combination administered to the subject comprises a dual inhibitor that targets both 5-LOX and COX-2. In some embodiments, the NOX inhibitor and the dual 5-LOX/COX-2 inhibitor are administered to the subject in a single dosage form, i.e., the NOX inhibitor and the dual 5-LOX/COX-2 inhibitor are administered as a combination drug. In other embodiments, the NOX inhibitor and the dual 5-LOX/COX-2 are administered to the subject in a multi-unit dosage form in which the subject is administered multiple, separate compositions, with each respective composition including one of the NOX inhibitor and the dual 5-LOX/COX-2 inhibitor. In some embodiments, the NOX inhibitor and the dual 5-LOX/COX-2 inhibitor combination is administered to the subject twice daily. In some embodiments, the NOX inhibitor and dual 5-LOX/COX-2 inhibitor combination is administered to the subject for at least two days.

Various dual 5-LOX/COX-2 inhibitors are known to those skilled in the art and include, but are not necessarily limited to, licofelone, darbufelone, CI-987, and various thiazoles and thiazolidinones as disclosed, for example, in Liaras, et al., (2018) Molecules, 23(3): 685. Various embodiments of the NOX inhibitor and dual 5-LOX and COX-2 inhibitor combination are contemplated herein, and each respective dual 5-LOX/COX-2 inhibitor identified above may be utilized in a different embodiment of the NOX inhibitor and dual 5-LOX and COX-2 inhibitor combination. Furthermore, embodiments of the NOX inhibitor and dual 5-LOX/COX-2 inhibitor combination utilized in the methods described herein can include one or more dual 5-LOX/COX-2 inhibitors.

In some embodiments, the NOX inhibitor of the NOX inhibitor and dual 5-LOX/COX-2 inhibitor combination is N-acetylcysteine. In some embodiments, the dual 5-LOX/COX-2 inhibitor of the NOX inhibitor and dual 5-LOX/COX-2 inhibitor combination is licofelone. As reflected in the Examples below, the administration of N-acetylcysteine in a dosage of 100 mg/kg in combination with zileuton (5 mg/kg) and celecoxib (10 mg/kg) has been found to reduce brain capillary leakage in in vivo suffering from seizures. In view of this and the teachings of U.S. Pat. No. 11,433,052 evidencing the efficacy of licofelone to reduce brain capillary leakage in subjects suffering from seizures when administered in a dosage of 5 mg/kg and 10 mg/kg, it is believed the administration of N-acetylcysteine in a dosage of about 100 mg/kg and licofelone in a dosage from about 5 mg/kg to about 10 mg/kg would also be effective in reducing brain capillary leakage, and thus treating subjects suffering from BBB dysfunction. Accordingly, administering an effective amount of the NOX inhibitor and dual 5-LOX/COX-2 inhibitor combination in the method for treating BBB dysfunction may comprise administering N-acetylcysteine in a dosage of about 100 mg/kg and administering licofelone in a dosage of about 5 mg/kg to about 10 mg/kg.

In some embodiments, the administration of the NOX inhibitor and dual 5-LOX/COX-2 inhibitor combination is effective in reducing brain capillary leakage in the subject.

As will be understood by those skilled in the art, BBB dysfunction is associated with various conditions impacting the brain, including, but not necessarily limited to seizure, epilepsy, Alzheimer's disease and/or dementia, Parkinson's disease, brain cancer, multiple sclerosis, stroke, brain trauma, an infectious disease of the brain (e.g., malaria), or peripheral inflammation or inflammation of the central nervous system (CNS). In this regard, the methods for treating BBB dysfunction disclosed herein can be useful in connection with a number of conditions. Accordingly, in some embodiments, the methods for treating BBB dysfunction in a subject may further involve identifying the subject as having seizures, epilepsy, Alzheimer's disease and/or dementia, Parkinson's disease, brain cancer, multiple sclerosis, stroke, brain trauma, an infectious disease of the brain, and/or peripheral inflammation or inflammation of the CNS.

Current treatment of Alzheimer's disease includes five drugs: three acetylcholine esterase inhibitors (donepezil, rivastigmine, galantamine), one NMDA receptor blocker (memantine), and two controversial monoclonal antibodies (aducanumab and lecanemab). Acetylcholine esterase inhibitors only work in a limited number of AD patients, and if they work, they lose their effect after 6-12 months. The same is true for the NMDA receptor blocker memantine. Not enough information is yet available on aducanumab and lecanemab, which were approved by the FDA in June 2021 under an accelerated approval pathway (aducanumab) and Jan. 6, 2023 (lecanemab). Both aducanumab and lecanemab, however, are extremely expensive (up to $50,000 per patient per year) and comes with significant adverse effects (brain swelling, brain bleeding). Importantly, the efficacy of aducanumab and lecanemab is still unclear. In addition, treatment of seizures/epilepsy in Alzheimer's disease patients is currently done with 3-4 ASDs that themselves come with significant adverse effects, including cognitive impairment. Thus, treatment of both Alzheimer's disease and epilepsy as well as the combination (Alzheimer's disease with epilepsy), is clinically extremely challenging. No treatment is believed to be available that improves any of the underlying symptoms.

As BBB dysfunction is associated with conditions impacting the brain, including seizures, and is known to be a cause and consequence of seizures in epilepsy, administration of the NOX inhibitor and one or more inhibitors for inhibiting 5-LOX and COX-2 disclosed herein may find particular utility in treating subjects with reoccurring seizures, such as subjects with epilepsy or subjects with Alzheimer's disease with epilepsy. In this regard, the presently disclosed subject matter also includes methods for treating recurrent seizures in which a combination of a NOX inhibitor and one or more inhibitors for inhibiting 5-LOX and COX-2 is administered to a subject suffering from recurrent seizures, such as a subject with epilepsy or Alzheimer's disease with epilepsy. Accordingly, in some embodiments the method for treating recurrent seizures further involves identifying the subject as having epilepsy or Alzheimer's disease with epilepsy.

An exemplary method for treating recurrent seizures in a subject involves administering a combination of a NOX inhibitor, a 5-LOX inhibitor, and a COX-2 inhibitor (or NOX/5-LOX/COX-2 inhibitor combination) to a subject suffering from recurrent seizures. Accordingly, in some embodiments of the method for treating recurrent seizures, the one or more inhibitors for inhibiting 5-LOX and COX-2 comprises two separate inhibitors: a 5-LOX inhibitor; and a COX-2 inhibitor. In some embodiments, the NOX/5-LOX/COX-2 inhibitor combination is administered to the subject in a single dosage form, i.e., the NOX inhibitor, the 5-LOX inhibitor, and the COX-2 inhibitor of the NOX/5-LOX/COX-2 inhibitor combination are administered as a combination drug. In other embodiments, the NOX/5-LOX/COX-2 inhibitor combination may be administered to the subject in a multi-dosage form in which the subject is administered multiple, separate compositions, with each composition including one or more of the NOX inhibitor, the 5-LOX inhibitor, and the COX-2 inhibitor of the NOX/5-LOX/COX-2 inhibitor combination. For instance, in some embodiments, the subject may be administered two separate compositions: a first composition comprising the 5-LOX inhibitor and the COX-2 inhibitor; and a second composition comprising the NOX inhibitor. In some embodiments, the subject may be administered three separate compositions: a first composition comprising the 5-LOX inhibitor; a second composition comprising the COX-2 inhibitor; and a third composition comprising the NOX inhibitor. In some embodiments, the NOX/5-LOX/COX-2 inhibitor combination is administered to the subject twice daily. In some embodiments, the NOX/5-LOX/COX-2 inhibitor combination is administered to the subject for at least two days.

One or more of the 5-LOX inhibitors, one or more of the COX-2 inhibitors, and one or more of the NOX inhibitors identified above for the methods of treating BBB dysfunction may be utilized in the NOX/5-LOX/COX-2 inhibitor combination in the methods for treating recurrent seizures. In some embodiments, the NOX inhibitor of the NOX/5-LOX/COX-2 inhibitor combination is N-acetylcysteine. In some embodiments, the 5-LOX inhibitor of the NOX/5-LOX/COX-2 inhibitor combination is zileuton. In some embodiments, the COX-2 inhibitor of the NOX/5-LOX/COX-2 inhibitor combination is celecoxib. In some embodiments, the NOX/5-LOX/COX-2 inhibitor combination comprises acetylcysteine, zileuton, and celecoxib. In some embodiments, administering the NOX/5-LOX/COX-2 inhibitor combination comprises administering N-acetylcysteine in a dosage of about 100 mg/kg, administering zileuton in a dosage of about 5 mg/kg, and administering celecoxib in a dosage of about 10 mg/kg.

In some embodiments, administration of the NOX/5-LOX/COX-2 inhibitor combination is effective in reducing BBB dysfunction in the subject suffering from recurrent seizures. In some embodiments, reduced BBB dysfunction is characterized by a reduction in brain capillary leakage. In some embodiments, administration of the NOX/5-LOX/COX-2 inhibitor combination is effective in reducing brain capillary leakage in the subject suffering from recurrent seizures.

Another exemplary method for treating recurrent seizures in a subject involves administering a combination comprising a NOX inhibitor and a dual 5-LOX/COX-2 inhibitor to a subject suffering from recurrent seizures, such as a subject with epilepsy or Alzheimer's disease with epilepsy. Accordingly, in some embodiments of the method for treating recurrent seizures, the one or more inhibitors for inhibiting 5-LOX and COX-2 comprises a dual inhibitor that targets both 5-LOX and COX-2. In some embodiments, the NOX inhibitor and the dual 5-LOX/COX-2 inhibitor are administered to the subject in a single dosage form, i.e., the NOX inhibitor and the dual 5-LOX/COX-2 inhibitor are administered as a combination drug. In other embodiments, the NOX inhibitor and the dual 5-LOX/COX-2 are administered to the subject in a multi-dosage form in which the subject is administered multiple, separate compositions, with each respective composition including one of the NOX inhibitor and the dual 5-LOX/COX-2 inhibitor. In some embodiments, the NOX inhibitor and the dual 5-LOX/COX-2 inhibitor combination is administered to the subject twice daily. In some embodiments, the NOX inhibitor and dual 5-LOX/COX-2 inhibitor combination is administered to the subject for at least two days.

One or more of the NOX inhibitors and one or more dual 5-LOX/COX-2 inhibitors identified above for the methods of treating BBB dysfunction may be utilized in the NOX inhibitor and dual 5-LOX/COX-2 inhibitor combination in the methods for treating recurrent seizures. In some embodiments, the NOX inhibitor of the NOX inhibitor and dual 5-LOX/COX-2 inhibitor combination is N-acetylcysteine. In some embodiments, the dual 5-LOX/COX-2 inhibitor of the NOX inhibitor and dual 5-LOX/COX-2 inhibitor combination is licofelone. As reflected in the Examples below, the administration of N-acetylcysteine in a dosage of 100 mg/kg in combination with zileuton (5 mg/kg) and celecoxib (10 mg/kg) has been found to reduce brain capillary leakage in in vivo suffering from seizures. In view of this and the teachings of U.S. Pat. No. 11,433,052 evidencing the efficacy of licofelone to reduce brain capillary leakage in subjects suffering from seizures when administered in a dosage of 5 mg/kg and 10 mg/kg, it is believed the administration of N-acetylcysteine in a dosage of about 100 mg/kg and licofelone in a dosage from about 5 mg/kg to about 10 mg/kg would also be effective in reducing brain capillary leakage, and thus treating the seizure-affected subject. Accordingly, administering the NOX inhibitor and dual 5-LOX/COX-2 inhibitor combination in the method of treating recurrent seizures may comprise administering N-acetylcysteine in a dosage of about 100 mg/kg and administering licofelone in a dosage of about 5 mg/kg to about 10 mg/kg.

In some embodiments, administration of the NOX inhibitor dual 5-LOX/COX-2 inhibitor combination is effective in reducing BBB dysfunction in the subject suffering from recurrent seizures. In some embodiments, administration of the NOX inhibitor dual 5-LOX/COX-2 inhibitor combination is effective in reducing brain capillary leakage in the subject suffering from recurrent seizures.

In some embodiments, the methods of treatment disclosed herein further involve administering an anti-seizure drug (ASD) to the subject.

In some embodiments, NOX/5-LOX/COX-2 inhibitor combinations and NOX inhibitor and dual 5-LOX/COX-2 inhibitor combinations disclosed herein may be administered with a pharmaceutically acceptable carrier.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

All patents, patent applications, published applications and publications, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently disclosed subject matter, representative methods, devices, and materials are described herein.

With respect to the therapeutic methods of the presently disclosed subject matter, a preferred subject is a mammal. A preferred mammal is a human. As used herein, the term “subject” includes both human and animal subjects. Thus, veterinary therapeutic uses are provided in accordance with the presently disclosed subject matter.

As used herein, the terms “treatment” or “treating” relate to any therapeutic treatment and/or prophylactic treatment to prevent development or reduce severity of a condition. The terms “treatment” or “treating” include: (1) preventing a condition; (2) inhibiting the condition, i.e., arresting the development of the condition; or (3) ameliorating or relieving the symptoms of the condition, i.e., causing regression of the condition.

As used herein, the term “pharmaceutically acceptable carrier” refers to a solid or liquid filler, diluent, and/or encapsulating substance that may be safely administered to a subject to facilitate delivery of a composition.

Administration of the NOX/5-LOX/COX-2 inhibitor combinations and NOX inhibitor and dual 5-LOX/COX-2 inhibitor combinations disclosed herein can occur intravenously, intramuscularly, intraperitoneally, or orally.

The present application can “comprise” (open-ended) or “consist essentially of” the components of the present invention as well as other ingredients or elements described herein.

As used herein, “comprising” is open-ended and means the elements recited, or their equivalent in structure or function, plus any other element or elements which are not recited. The terms “having” and “including” are also to be construed as open-ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about”, when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

As used herein, ranges can be expressed as from “about” one particular value, and/or to “about” another particular value. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently disclosed subject matter is further illustrated by the following specific but non-limiting examples. The following examples may include compilations of data that are representative of data gathered at various times during the course of development and experimentation related to the present invention.

### EXAMPLES

The studies described in these Examples examined the effect of NOX, 5-LOX, and COX-2 inhibition on BBB leakage in two separate in vivo models. More particularly, the studies described in these Examples examined the effect of N-acetylcysteine (NAC), zileuton (ZLT), and celecoxib (CEL) in combination on BBB leakage in two separate in vivo models for AD with epilepsy.

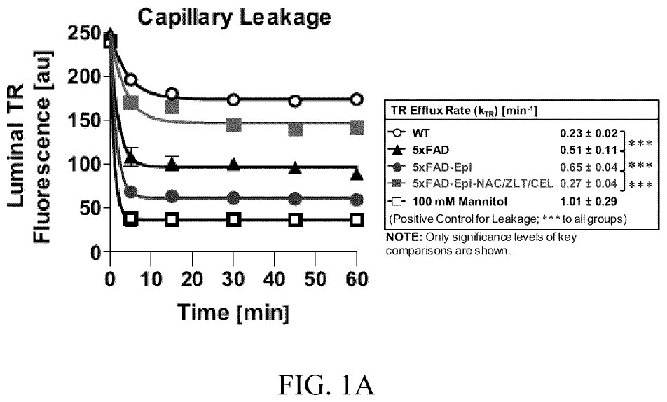

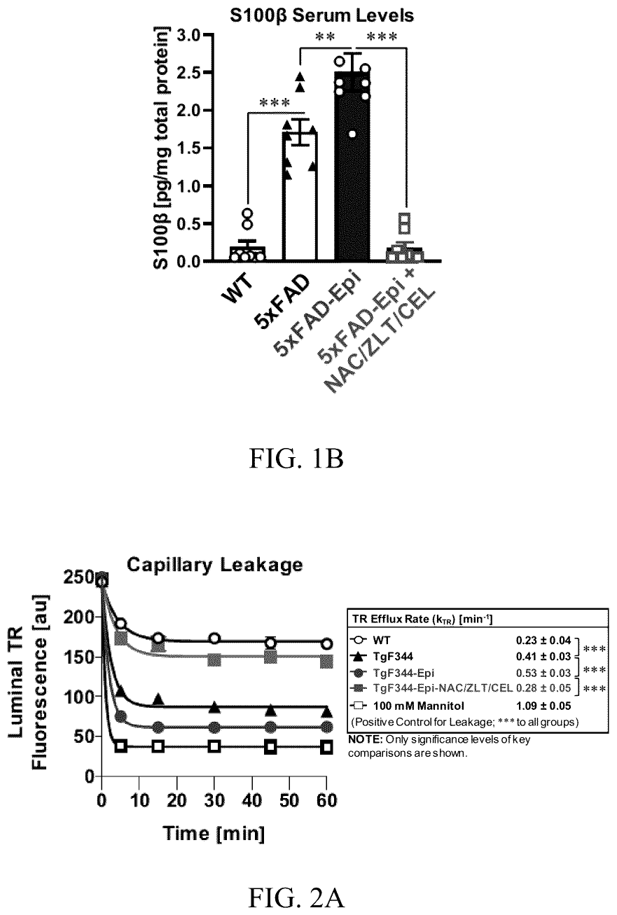

With reference to FIGS. 1A and 1i, the effect of NOX/5-LOX/COX-2 inhibition on BBB leakage in 5×FAD mice with epilepsy (5×FAD-Epi) treated with NAC, ZLT, and CEL in combination as compared to untreated 5×FAD and 5×FAD-Epi mice was examined. 5×FAD mice (breeding colony) underwent seizure induction with kainic acid to generate 5×FAD-Epi mouse models. Wild-type: littermate controls. S1000 is an astrocytic protein that has emerged as a potential biomarker for barrier leakage in epilepsy. Average kainic acid dosage was 33.5±2.179 mg/kg. Average diazepam dose was 13.250±1.218 mg/kg. Status epilepticus (SE) (condition of continuous seizure activity) was developed in 17 out of 20 mice tested, with one mouse dying after SE. The 16 surviving SE mice were randomly divided into two groups of treatment (n=8) and vehicle (n=8).

As reflected in the results shown in FIGS. 1A and 1, treating 5×FAD-Epi mice models with the combination of NAC, ZLT, and CEL (100/5/10 mg/kg NAC/ZLT/CEL every 12 hours for two days, intraperitoneal injection) fully abolished seizure-mediated capillary leakage and S1000 levels in vivo as compared to the untreated 5×FAD and 5×FAD-Epi mice.

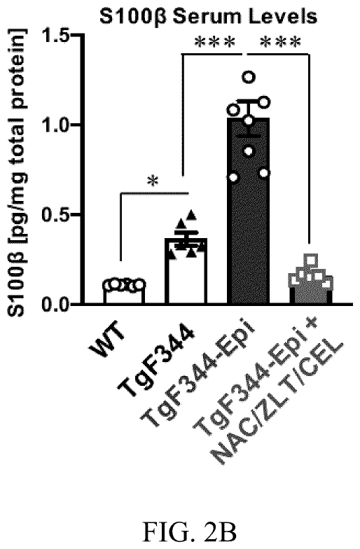

With reference to FIGS. 2A and 2B, the effect of NOX/5-LOX/COX-2 inhibition on BBB leakage in TgF344 rats with epilepsy (TgF344-Epi) treated with NAC, ZLT, and CEL in combination as compared to untreated TgF344 and TgF344-Epi rats was examined. TgF344 rats (Rat Resource and Research Center (RRRC), Columbia, MO) underwent seizure induction with pilocarpine to generate TgF344-Epi rat models. Wild-type: littermate controls. S1000 is an astrocytic protein that has emerged as a potential biomarker for barrier leakage in epilepsy. Average pilocarpine dosage was 31.11±1.11 mg/kg. Average diazepam dose was 18.9±4.4 mg/kg. Although not designated with the identifier “AD” herein or in the drawings, it should be appreciated that it is well-known that TgF344 rats are models for Alzheimer's disease. SE was developed in all 18 rats tested; 4 animals died after seizure induction. The remaining 14 SE mice were randomly divided into two groups of treatment (n=6) and vehicle (n=8).

As reflected in the results shown in FIGS. 2A and 2B, treating TgF344-Epi rat models with the combination of NAC, ZLT, and CEL (100/5/10 mg/kg NAC/ZLT/CEL every 12 hours for two days, intraperitoneal injection) significantly reduced seizure-mediated capillary leakage and S100β levels in vivo as compared to the untreated TgF344 and TgF344-Epi rats.

Collectively, the data in FIGS. 1A, 1B, 2A, and 2B provide evidence that 5-LOX/COX-2 inhibition combined with the reactive oxygen species scavenger NAC could repair BBB leakage in subjects with Alzheimer's disease with epilepsy.

All publications, patents, and patent applications mentioned in this specification are herein incorporated by reference to the same extent as if each individual publication, patent, or patent application was specifically and individually indicated to be incorporated by reference.

It will be understood that various details of the presently disclosed subject matter can be changed without departing from the scope of the subject matter disclosed herein. Furthermore, the foregoing description is for the purpose of illustration only and not for the purpose of limitation.

What is claimed is:

1. A method for treating blood-brain barrier (BBB) dysfunction in a subject, comprising: administering to the subject an effective amount of a combination of a nicotinamide adenine dinucleotide phosphate (NADPH) oxidase (NOX) inhibitor and one or more inhibitors for inhibiting 5-lipoxygenase (5-LOX) and cyclooxygenase-2 (COX-2) to reduce BBB leakage.

2. The method of claim 1, wherein the NOX inhibitor is N-acetylcysteine.

3. The method of claim 1, wherein the one or more inhibitors for inhibiting 5-LOX and COX-2 comprises a 5-LOX inhibitor and a COX-2 inhibitor.

4. The method of claim 3, wherein the 5-LOX inhibitor is zileuton.

5. The method of claim 3, wherein the COX-2 inhibitor is celecoxib.

6. The method of claim 3, wherein the NOX inhibitor is N-acetylcysteine, the 5-LOX inhibitor is zileuton, and the COX-2 inhibitor is celecoxib.

7. The method of claim 6, wherein administering the effective amount of the combination comprises administering N-acetylcysteine in a dosage of about 100 mg/kg, administering zileuton in a dosage of about 5 mg/kg, and administering celecoxib in a dosage of about 10 mg/kg.

8. The method of claim 1, wherein the one or more inhibitors for inhibiting 5-lipoxygenase (5-LOX) and cyclooxygenase-2 (COX-2) comprises a dual 5-LOX/COX-2 inhibitor.

9. The method of claim 8, wherein the dual 5-LOX/COX-2 inhibitor is licofelone.

10. The method of claim 1, wherein the combination is administered to the subject in a single dosage form.

11. The method of claim 1, wherein the combination is administered in a multi-dosage form.

12. The method of claim 1, wherein the combination is administered to the subject twice daily for at least two days.

13. The method of claim 1, and further comprising:
identifying the subject as having seizures, epilepsy, Alzheimer's disease and/or dementia, Parkinson's disease, brain cancer, multiple sclerosis, stroke, brain trauma, an infectious disease of the brain, and/or peripheral inflammation or inflammation of the central nervous system (CNS).

14. The method of claim 1, wherein reduced BBB leakage is characterized by a reduction in brain capillary leakage.

15. A method for treating recurrent seizures, comprising: administering a combination of a nicotinamide adenine dinucleotide phosphate (NADPH) oxidase (NOX) inhibitor and one or more inhibitors for inhibiting 5-lipoxygenase (5-LOX) and cyclooxygenase-2 (COX-2) to a subject in need thereof.

16. The method of claim 15, and further comprising identifying the subject as having Alzheimer's disease with epilepsy.

17. The method of claim 15, wherein the NOX inhibitor is N-acetylcysteine.

18. The method of claim 15, wherein the one or more inhibitors for inhibiting 5-LOX and COX-2 comprises a 5-LOX inhibitor and a COX-2 inhibitor.

19. The method of claim 18, wherein the 5-LOX inhibitor is zileuton.

20. The method of claim 18, wherein the COX-2 inhibitor is celecoxib.

21. The method of claim 18, wherein the NOX inhibitor is N-acetylcysteine, the 5-LOX inhibitor is zileuton, and the COX-2 inhibitor is celecoxib.

22. The method of claim 21, wherein administering the combination comprises administering N-acetylcysteine in a dosage of about 100 mg/kg, administering zileuton in a dosage of about 5 mg/kg, and administering celecoxib in a dosage of about 10 mg/kg.

23. The method of claim 15, wherein the one or more inhibitors for inhibiting 5-lipoxygenase (5-LOX) and cyclooxygenase-2 (COX-2) comprises a dual 5-LOX/COX-2 inhibitor.

24. The method of claim 23, wherein the dual 5-LOX/COX-2 inhibitor is licofelone.

25. The method of claim 15, wherein the combination is administered to the subject in a single dosage form.

26. The method of claim 15, wherein the combination is administered in a multi-dosage form.

27. The method of claim 15, wherein the combination is administered to the subject twice daily for at least two days.

28. The method of claim 15, and further comprising administering an anti-seizure drug (ASD).
